(19) United States
(12) Patent Application Publication
(10) Pub. No.: US 2002/0076442 A1
(43) Pub. Date: Jun. 20, 2002

(54) VITAMIN D3 ANALOG LOADED POLYMER FORMULATIONS FOR CANCER AND NEURODEGENERATIVE DISORDERS

(76) Inventors: MARTIN BURKE, BALTIMORE, MD (US); MARIA-CHRISTINA WHITE, BALTIMORE, MD (US); MARK C. WATTS, GRAND BLANC, MI (US); JAE KYOO LEE, SAN DIEGO, CA (US); BETTY M. TYLER, LAUREL, MD (US); GARY H. POSNER, BALTIMORE, MD (US); HENRY BREM, LUTHERVILLE, MD (US)

Correspondence Address: BANNER & WITCOFF, 1001 G STREET N W, SUITE 1100, WASHINGTON, DC, 20001, US

(21) Appl. No.: 09/145,867

(22) Filed: Sep. 2, 1998

Related U.S. Application Data
(60) Provisional application No. 60/057,436, filed on Sep. 2, 1997.

(51) Int. Cl. A61K 31/56; A61K 9/14

(52) U.S. Cl. 424/486; 514/179; 424/426; 424/428

(57) ABSTRACT

Localized delivery of 1,25 D₃ directly to a target area using biodegradable polymeric matrices maximizes the efficacy of this drug while minimizing systemic exposure and toxicity. Anticalcemic analogs of 1,25 D₃ have also been incorporated into controlled release polymer formulations to achieve efficacious intracranial concentrations of 1,25 D₃ analogs for the treatment of intracranial tumors as well as neurodegenerative disorders such as Alzheimer's disease as well as to maximize the efficacy of these analogs in the treatment of systemic malignancies. The therapeutic efficacy of these formulations was demonstrated through a variety of studies in vitro and in vivo. Hybrid analogs of 1,25 D₃ were incorporated into biodegradable polymer wafers composed of a polyanhydride copolymer of 1,3-bis(p-carboxyphenoxy)propane (CPP) and sebacic acid (SA) in a 20:80 molar ratio. In addition to providing improved treatments for malignancies and neurodegenerative disorders. the spatial localization and high reproducibility of this controlled delivery methodology presents a unique opportunity to study in vivo the poorly understood mechanisms of 1,25 D₃'s antiangiogenic, antiproliferative, and transcriptional regulating activities.

This application claims priority to U.S. Ser. No. 60/057,436 entitled “Controlled Release Vitamin D3 Derivative Formulations for Treatment of Cancer” filed Sep. 2, 1997 by Martin Burke, Maria-Christina White, Jau Kyoo Lee, Mark Watts, Betty M. Tyler, Gary Posner, and Henry Brem.

The United States Government has certain rights in this invention by virtue of National Institutes of Health grant No. CA 44530.

The Therapeutic Potential of 1,25-Dihydroxyvitamin D₃

### BACKGROUND OF THE INVENTION

The role of the seco-steroid hormone 1,25-Dihydroxyvitamin D₃ (1,25 D₃) in the regulation of calcium homeostasis and bone metabolism via action in the intestine, bone, kidney, and parathyroid glands has long been known. Recently, however, as the understanding of the endocrinological impact of 1,25 D₃ endocrinological impact has broadened, a variety of new potentially therapeutic roles have emerged. These include the treatment of a wide variety of neoplastic diseases, as well as neurodegenerative disorders of the central nervous system (CNS).

A potential role for 1,25 D₃ in the treatment of cancer was first suggested by epidemiological studies carried out in the 1980s and early 1990s which demonstrated a relationship between sunlight exposure. serum 1,25 D₃ levels, and the risk for fatal colon, breast, and prostate cancer (Garland, et al. Lancet 2:1176-1178 (1989): Garland, et al. Prev. Med. 19:614-622 (1990); Schwartz and Hulka Anticancer Res. 10:1307-1311 (1990)). Since that time, many researchers have demonstrated that 1,25 D₃ exerts potent antiproliferative and/or pro-differentiating activity on a wide variety of malignant cell types in vitro including colon, breast, prostate, hematopoietic cells, bone, lung, skin, and brain (Hulla, et al. Int. J. Cancer. 62:711-716; Elstner, et al. Cancer Res. 55:2822-2830 (1995); Peehl, Cancer Res. 54:805-810 (1994); Xu, et al. Exp. Cell Res. 214:250-257 (1993); van den Bemd, et al. J. Steroid Biochem. Mol. Biol. 55:337-346 (1995): Colston, et al. Lancet. 1:188-191 (1989); Naveilhan, et al. J. Neurosci. Res. 37:271-277 (1994)). Furthermore, 1,25 D₃ demonstrates highly potent anti-angiogenic activity in various model systems. (Oikawa, et al. Eur. J. Pharm. 178:247-250 (1990); Majewski, et al. Cancer Let. 75:35-39 (1993)). Metastases inhibition and chemopreventative actions have been revealed as well (Hansen, et al. Clin. Exp. Metastasis. 12:195-202 (1994)). Believed to be the result of a combination of these anticancer activities, 1,25 D₃-mediated solid tumor growth inhibition has been demonstrated in a variety of murine models of malignancy (Chiba, et al. Cancer Res. 45:5426-5430 (1985); Eisman, et al. Cancer Res. 47:21-25 (1987); Colston, et al. Lancet 1:188-191 (1989); Tsuchiya, et al. J. Orthop. Res. 11:122,130 (1993)). However, these potentially therapeutic activities of 1,25 D₃ are strictly limited by the causation of toxic hypercalcemia at supraphysiological dosing regimens (Vieth, et al. Bone Miner. 11:267-272 (1990)). As a result, the small number of oncological clinical trials with 1,25 D₃ completed to date have demonstrated a high incidence of dose-limiting hypercalcemia and failed to show substantial antitumor efficacy (Cinningham, et al. Br. Med. J. 291:1153-1155 (1985); Koeffler, et al. Cancer Treat. Rep. 69:1399-1407 (1985); Kelsey, et al. Lancet 340:316-317 (1992)).

Due to its demonstrated ability to upregulate Nerve Growth Factor (NGF), a neurotrophic factor crucial to the maintenance of proper cholinergic nerve function in the basal forebrain, hippocampus, and cortex, 1,25 D₃ has also been implicated in the treatment of Alzheimer's disease. However, due to its limited penetration of the blood brain barrier (BBB) and toxic systemic hypercalcemic effects, attempts to upregulate in the brain by delivering 1,25D₃ systemically have been unsuccessful (Saporito. et al. Experimental Neurology, 123: 295-302, 1993). To bypass the BBB and reveal the therapeutic potential of 1,25D₃ in the treatment of Alzheimer's, mini-osmotic pumps have been utilized to deliver the drug into the murine brain intracerebroventricularly (i.c.v.). (Carswell, S. Vitamin D in the Nervous System: Actions and Therapeutic Potential. Vitamin D: 1197-1211, 1997; Saporito, et al. Brain Research, 633: 189-196, 1994). Although no NGF mRNA upregulation was observed following a single injection of 1,25D₃ into the brain, pump-mediated chronic delivery for 6 days resulted in pharmacologically relevant upregulation of NGF in cholinergic neurons. The success of this treatment, however, was limited since i.c.v. administration also results in high systemic concentrations of 1,25 D₃ leading to dose-limiting toxic hypercalcemia. Furthermore, the clinical application of this pump-mediated delivery system is perturbed by a high incidence of infection and blockage of the catheter system.

To date, the most successful strategy for enhancing the therapeutic index of 1,25 D₃ has been the design and synthesis of unnatural structural analogs with the objective of separating undesirable calcitropic activity from potentially therapeutic anti-angiogenic, antiproliferative, and transcriptional regulating activities (Elstner, et al. Cancer. Res. 55:2822-2830 (1995); Zhou and Norman Endocrinology, 36:1145-1152 (1995)). Several hundred 1,25 D₃ analogs have been prepared and tested worldwide, some of which appear successful in achieving this goal in pre-clinical studies and are currently undergoing small-scale clinical evaluation in the United States. The Posner group at Johns Hopkins University has developed a methodology for separating 1,25 D₃'s desired and undesired activities which invokes the coupling of various powerful antiproliferative enhancing structural units on the C,D-ring side chain with an anticalcemic 1-b-hydroxymethyl A-ring modification (Posner, et al. J. Org. Chem., 62: 3299-3314, 1997; Posner, et al. J. Med. Chem., 35: 3280, 1992; Posner, et al. Bioorganic Medicinal Chemistry Letters, 4: 2919, 1994). This strategy has yielded promising new hybrid analogs that demonstrate retained antiproliferative activity in vitro and dramatically minimized calcemic effects in vivo relative to 1,25 D₃.

It is an object of this invention to provide vitamin D3 formulations for treatment of cancer with reduced toxicity.

It is a further object of this invention to provide vitamin D3 formulations useful in treatment of neurodegenerative disorders.

### SUMMARY OF THE INVENTION

Localized delivery of 1,25 D₃ directly to a target area using biodegradable polymeric matrices maximizes the efficacy of this drug while minimizing systemic exposure and toxicity. Anticalcemic analogs of 1,25 D₃ have also been incorporated into controlled release polymer formulations to achieve efficacious intracranial concentrations of 1,25 D₃ analogs for the treatment of intracranial tumors as well as neurodegenerative disorders such as Alzheimer's disease as well as to maximize the efficacy of these analogs in the treatment of systemic malignancies. In addition to providing improved treatments for malignancies and neurodegenerative disorders, the spatial localization and high reproducibility of this controlled delivery methodology presents a unique opportunity to study in vivo the poorly understood mechanisms of 1,25 D₃'s antiangiogenic, antiproliferative, and transcriptional regulating activities.

The therapeutic efficacy of these formulations was demonstrated through a variety of studies in vitro and in vivo. Hybrid analogs of 1,25 D₃ were incorporated into biodegradable polymer wafers composed of a polyanhydride copolymer of 1,3-bis(p-carboxyphenoxy)propane (CPP) and sebacic acid (SA) in a 20:80 molar ratio. Various drug/polymer combinations were co-dissolved in an organic solvent followed by drying in vacuo. The resulting homogenous drug/polymer formulation was then compression molded into cylindrical wafers using a miniature custom made compression molding device, similar to micro KBr dies available from Aldrich. Following systemic or intracranial implantation of drug loaded polymer wafers, surface erosion of the polymer matrix over a period of two to three weeks led to sustained release of these novel therapeutic agents to a specific site within the body.

The results demonstrate that these drugs are potent inhibitors of proliferation against a variety of murine tumor cell lines in vitro. Strengthening the rationale for sustained drug delivery, a proportional relationship between antiproliferative activity and exposure time was shown. Evidencing therapeutic potential in the treatment of neurodegenerative disorders such as Alzheimer's disease, studies demonstrated that the 1,25 D₃ analog MCW-YB can significantly upregulate the synthesis of NGF by murine L929 fibroblasts in vitro. The two most potent 1,25 D₃ analogs demonstrate dramatically reduced calcemic activity when compared to the parent compound. The most potent hybrid analogs were also successfully loaded into biodegradable polyanhydride copolymer wafers, and the sustained release of these compounds from polymer wafers was demonstrated in vivo. These 1,25 D₃ analog-loaded polymer wafers were well tolerated in the murine brain and flank at drug loading doses ranging from 0.1 to 1% by weight. Intracranial implantation of 5 mg pCPP:SA(20:80) polymer wafers loaded with the 1,25 D₃ analog JK-1626-2 or MCW-YB at 0.1% by weight resulted in no significant weight loss or rises in blood ionized calcium levels for 7 days. Similar implantation of 0.5% MCW-YB-loaded wafers into Sprague-Dawley rats yielded no weight loss or rise in serum ionized calcium for up to 12 days. Furthermore, the site-specific polymeric delivery of 1,25 D₃ analogs to the brain results in diminished systemic hypercalcemia when compared to polymeric delivery to the flank. Collectively, these studies reveal that sustained delivery via biodegradable polymers of 1,25 D₃ hybrid analogs are useful for the treatment for several types of systemic and CNS malignancies, as well as neurodegenerative disorders.

### BRIEF DESCRIPTION OF THE DRAWINGS

FIG. 1 is a graph of the antiproliferative activity of 1,25 D₃ and hybrid analogs at concentrations of 1, 10, 100, and 1000 nM against murine B16 malignant melanoma cells. Results are expressed as % of control, the mean cell number from 6 wells for each drug concentration divided by the mean cell number from 6 control wells receiving only solvent (isopropanol).

FIG. 2 is a graph of the antiproliferative activity of 1,25 D₃ and hybrid analogs at 1, 10, 100 and 1000 nM against murine EMT6 breast carcinoma cells. Results are expressed as % of control, the mean cell number from 6 wells for each drug concentration divided by the mean cell number from 6 control wells receiving only solvent (isopropanol).

FIG. 3 is a graph of the antiproliferative activity of 1,25 D₃ and hybrid analogs at 1, 10, 100 and 1000 nM against murine RENCA renal cell carcinoma cells. Results are expressed as % OF CONTROL, the mean cell number from 6 wells for each drug concentration divided by the mean cell number from 6 control wells receiving only solvent (isopropanol).

FIG. 4 is a graph of the exposure time dependent antiproliferative activity of 1,25 D₃ at 10 μM against B16 malignant melanoma cells. Results are expressed as % of control, the mean cell number from 3 wells for each drug concentration divided by the mean cell number from 3 control wells receiving only solvent (0.4% isopropanol).

### DETAILED DESCRIPTION OF THE INVENTION

Polymer-mediated delivery of 1,25 D₃ or analogs thereof directly to an intracranial target has several advantages including circumvention of the blood brain barrier (BBB), achievement of high drug concentrations in a desired locus, sustained drug delivery for up to five years, and minimal systemic exposure and toxicity. Systemic application of this polymer-based delivery strategy also offers the advantage of maintaining constant, high levels of drug in a peripheral target area with a smaller overall dose. The combination of controlled release polymer formulations with analogs of 1,25 D₃ characterized by low calcemic activity and maintained therapeutic activities provides additional advantages for treatment with both systemic and neurological malignancies as well as neurodegenerative disorders such as Alzheimer's disease.

I. Compositions

Vitamin D3 and D3 Analogs

D3 Analogs having anti-proliferative activity can be delivered using controlled and/or sustained release formulations for treatment of cancer. These have the following general and specific formulas and are described by Posner, et al. J. Org. Chem., 62: 3299-3314, 1997; Posner, et al. J. Med. Chem., 35: 3280, 1992; Posner, et al. Bioorganic Medicinal Chemistry Letters, 4: 2919, 1994, the contents of which are hereby incorporated by reference.

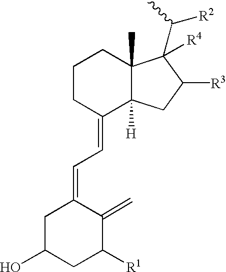

wherein R¹ is —OH or CH₂OH, R2 is a C4-6 chain or a C4-6 alkoxy chain, wherein the chain includes one or more substituents selected from the group consisting of hydroxyl groups, preferably tertiary hydroxyl groups, alkene groups, alkyne groups, alkyl groups, preferably methyl and ethyl, and ketones, and R3 and R4 are either H or together form a double bond. The formula is also intended to include fluorinated derivatives, with fluorines at one or more of the positions shown in U.S. Pat. Nos. 5,428,029, 5,612,328, 5,039,671, and 5,451,574, the contents of which are hereby incorporated by reference.

Preferred compounds are 1,25 D₃ and five hybrid analogs with an anticalcemic 1-b-hydroxymethyl A-ring modification (JK-III-7-2, JK-132-2, JK-1626-2, MCW-005-YB, MCW-068-Y-EE).

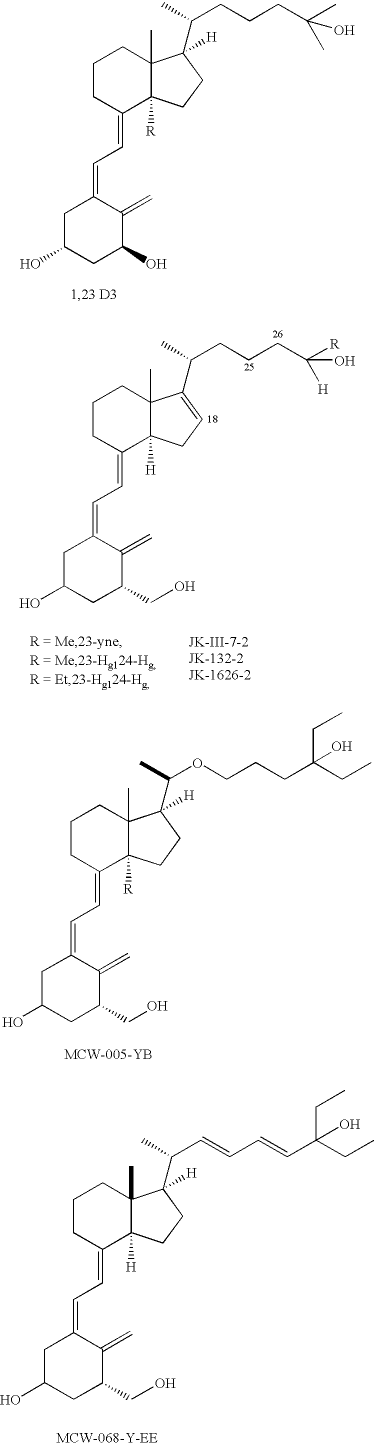

The structures of 1,25 D₃ and five hybrid analogs synthesized by Gary Posner et. al. (JK-III-7-2, JK-132-2, JK-1626-2, MCW-005-YB, MCW-068-Y-EE). Other analogs are known, for example, as described by Elstner, et al. Cancer. Res. 55:2822-2830 (1995); Zhou and Norman Endocrinology, 36:1145-1152 (1995)).

Controlled and/or Sustained Release Formulations

The Vitamin D3 derivatives are administered in controlled and/or sustained release formulations. These can further include a pharmaceutically acceptable carrier such as saline, phosphate buffered saline, cells transduced with a gene encoding other bioactive molecules, microparticles, or other conventional vehicles.

i. Polymeric Formulations

The Vitamin D3 derivatives can be encapsulated into a biocompatible polymeric matrix, most preferably biodegradable. The Vitamin D3 derivative are preferably released by diffusion and/or degradation over a therapeutically effective time, for example, between eight hours to five years, more typically betwee one week and one year, depending on the indication. As used herein, microencapsulated includes incorporated onto or into or on microspheres, microparticles, or microcapsules. Microcapsules is used interchangeably with microspheres and microparticles, although it is understood that those skilled in the art of encapsulation will recognize the differences in formulation methods, release characteristics, and composition between these various modalities. The microspheres can be directly implanted or delivered in a physiologically compatible solution such as saline.

Biocompatible polymers can be categorized as biodegradable and non-biodegradable. Biodegradable polymers degrade in vivo as a function of chemical composition, method of manufacture, and implant structure. Synthetic and natural polymers can be used although synthetic polymers may be preferred due to more uniform and reproducible degradation and other physical properties. Examples of synthetic polymers include polyanhydrides, polyhydroxyacids such as polylactic acid, polyglycolic acid and copolymers thereof, polyesters, polyamides, polyorthoesters, and some polyphosphazenes. Examples of naturally occurring polymers include proteins and polysaccharides such as collagen, hyaluronic acid, albumin and gelatin. The ideal polymer must be processible and flexible enough so that it does not crumble or fragment during use.

Vitamin D3 derivatives and optionally, other drugs or additives, can be encapsulated within, throughout, and/or on the surface of the implant. The Vitamin D3 derivative is released by diffusion, degradation of the polymer, or a combination thereof. There are two general classes of biodegradable polymers: those degrading by bulk erosion and those degrading by surface erosion. The latter polymers are preferred where more linear release is required. The time of release can be manipulated by altering chemical composition; for example, by increasing the amount of an aromatic monomer such as p-carboxyphenoxy propane (CPP) which is copolymerized with a monomer such as sebacic acid (SA). A particularly preferred polymer is CPP-SA (20:80). Use of polyanhydrides in controlled delivery devices has been reported by Leong, et al., J. Med. Biomed. Mater. Res., 19:941 (1985); J. Med. Biomed. Mater. Res., 20:51 (1986); and Rosen, et al., Biomaterials, 4:131 (1983). U.S. Patents that describe the use of polyanhydrides for controlled delivery of substances include U.S. Pat. No. 4,857,311 to Domb and Langer, U.S. Pat. No. 4,888,176 to Langer, et al., and U.S. Pat. No. 4,789,724 to Domb and Langer. Other polymers such as polylactic acid, polyglycolic acid, and copolymers thereof have been commercially available as suture materials for a number of years and can be readily formed into devices for drug delivery.

Non-biodegradable polymers remain intact in vivo for extended periods of time (years). Agents loaded into the non-biodegradable polymer matrix are released by diffusion through the polymer's micropore lattice in a sustained and predictable fashion, which can be tailored to provide a rapid or a slower release rate by altering the percent Vitamin D3 derivative loading, porosity of the matrix, and implant structure. Ethylene-vinyl acetate copolymer (EVAc) is an example of a nonbiodegradable polymer that has been used as a local delivery system for proteins and other macromolecules, as reported by Langer, R., and Folkman, J., Nature (London), 263:797-799 (1976). Others include polyurethanes, polyacrylonitriles, and some polyphosphazenes.

In the preferred embodiment, only polymer and Vitamin D3 derivatives to be released are incorporated into the delivery device, although other biocompatible, preferably biodegradable or metabolizable, materials can be included for processing purposes as well as additional therapeutic agents.

Although not the preferred embodiment, polymeric gel formulations can also be used to administer the drug. Many suitable polymeric materials are known, including polyoxyethylene block compolymers such as the Pluronics™ and Poloxamers™ marketed by BASF, photopolymerizable gels such as those described by U.S. Pat. No. 5,573,934 to Hubbell, et al.

ii. Additives

Buffers, acids and bases can be used to adjust the pH of the composition. Agents to increase the diffusion distance of agents released from the implanted polymer can also be included.

Fillers are water soluble or insoluble materials incorporated into the formulation to add bulk. Types of fillers include sugars, starches and celluloses. The amount of filler in the formulation will typically be in the range of between about 1 and about 90% by weight.

Spheronization enhancers facilitate the production of spherical implants. Substances such as zein, microcrystalline cellulose or microcrystalline cellulose co-processed with sodium carboxymethyl cellulose confer plasticity to the formulation as well as implant strength and integrity. During spheronization, extrudates that are rigid, but not plastic, result in the formation of dumbbell shaped implants and/or a high proportion of fines. Extrudates that are plastic, but not rigid, tend to agglomerate and form excessively large implants. A balance between rigidity and plasticity must be maintained. The percent of spheronization enhancer in a formulation depends on the other excipient characteristics and is typically in the range of 10 to 90% (w/w).

Disintegrants are substances which, in the presence of liquid, promote the disruption of the implants. The function of the disintegrant is to counteract or neutralize the effect of any binding materials used in the formulation. The mechanism of disintegration involves, in large part, moisture absorption and swelling by an insoluble material. Examples of disintegrants include croscarmellose sodium and crospovidone which are typically incorporated into implants in the range of 1 to 20% of total implant weight. In many cases, soluble fillers such as sugars (mannitol and lactose) can also be added to facilitate disintegration of the implants.

Surfactants may be necessary in implant formulations to enhance wettability of poorly soluble or hydrophobic materials. Surfactants such as polysorbates or sodium lauryl sulfate are, if necessary, used in low concentrations, generally less than 5%.

Binders are adhesive materials that are incorporated in implant formulations to bind powders and maintain implant integrity. Binders may be added as dry powder or as solution. Sugars and natural and synthetic polymers may act as binders. Materials added specifically as binders are generally included in the range of about 0.5 to 15% w/w of the implant formulation. Certain materials, such as microcrystalline cellulose, also used as a spheronization enhancer, also have additional binding properties.

Various coatings can be applied to modify the properties of the implants. Three types of coatings are seal, gloss and enteric. The seal coat prevents excess moisture uptake by the implants during the application of aqueous based enteric coatings. The gloss coat improves the handling of the finished product. Water-soluble materials such as hydroxypropyl cellulose can be used to seal coat and gloss coat implants. The seal coat and gloss coat are generally sprayed onto the implants until an increase in weight between about 0.5% and about 5%, preferably about 1% for seal coat and about 3% for a gloss coat, has been obtained.

Enteric coatings consist of polymers which are insoluble in the low pH (less than 3.0) of the stomach, but are soluble in the elevated pH (greater than 4.0) of the small intestine. Polymers such as Eudragit*, RohmTech, Inc., Malden, Mass., and Aquateric*, FMC Corp., Philadelphia, Pa., can be used and are layered as thin membranes onto the implants from aqueous solution or suspension. The enteric coat is generally sprayed to a weight increase of about one to about 30%, preferably about 10 to about 15%, and can contain coating adjuvants such as plasticizers, surfactants, separating agents that reduce the tackiness of the implants during coating, and coating permeability adjusters. Other types of coatings having various dissolution or erosion properties can be used to further modify implant behavior. Such coatings are readily known to one of ordinary skill in the art.

iii. Manufacture of Controlled Release Devices

Controlled release devices are typically prepared in one of several ways. The polymer can be melted, mixed with the substance to be delivered, and then solidified by cooling. Such melt fabrication processes require polymers having a melting point that is below the temperature at which the substance to be delivered and polymer degrade or become reactive. Alternatively, the device can be prepared by solvent casting, where the polymer is dissolved in a solvent, and the substance to be delivered is dissolved or dispersed in the polymer solution. The solvent is then evaporated, leaving the substance in the polymeric matrix. Solvent casting requires that the polymer be soluble in organic solvents and that the agents to be encapsulated be soluble or dispersible in the solvent. Similar devices can be made by solvent removal, phase separation or emulsification or even spray drying techniques. In still other methods, a powder of the polymer is mixed with the Vitamin D3 derivative and then compressed to form an implant.

Methods of producing implants also include granulation, extrusion, and spheronization. A dry powder blend is produced including the desired excipients and microspheres. The dry powder is granulated with water or other non-solvents for microspheres such as oils and passed through an extruder forming “strings” or “fibers” of wet massed material as it passes through the extruder screen. The extrudate strings are placed in a spheronizer which forms spherical particles by breakage of the strings and repeated contact between the particles, the spheronizer walls and the rotating spheronizer base plate. The implants are dried and screened to remove aggregates and fines. These methods can be used to make micro-implants (microparticles, microspheres, and microcapsules encapsulating Vitamin D3 derivatives to be released), slabs or sheets, films, tubes, and other structures.

II. Methods of Treatment

In the preferred embodiment the formulations are administered in a tumor or other sites to be treated, most preferentially intracranially. The dosage and formulation will be determined by the disorder to be treated. More or less of the polymeric material, or the polymer loading, can be used to treat the patient.

1,25 D₃ analogs can also be administered in combination with other chemotherapeutic agents such as cisplatin, BCNU, taxol, or cytokines such as IL-2 to potentiate the effects of locally delivered cytotoxic agents against solid tumors, alone or in combination with other types of local or targeted or systemic therapy such as radiation. Drug combinations for the treatment of neurodegenerative disorders can also be used.

The spatial localization and high reproducibility of this controlled delivery methodology also allows the study in vivo of the poorly understood mechanisms of 1,25 D₃'s antiangiogenic, antiproliferative, and transcriptional regulating activities.

### EXAMPLES

The present invention will be further understood by reference to the following non-limiting examples.

### Example 1

Testing the Antiproliferative Activity of 1,25 D₃ Hybrid Analogs Against a Series of Murine Malignant Cell Lines in vitro

Concentration Dependence in Proliferation Assays

In vitro proliferation assays were performed to measure the activity of 1,25 D₃ and its analogs against four murine metastatic tumor cell lines, B16 (malignant melanoma), RENCA (renal cell carcinoma), EMT6 (breast cell carcinoma), CT26 (colon carcinoma). All cell lines were grown and propagated in RPMI medium at 37° C. in 5% CO₂. Cultured cells were trypsinized and plated in triplicate at 10,000 cells/well in Falcon 24 well tissue culture plates. After 24 hours of incubation the cells received fresh media containing either solvent (isopropanol) or drug at concentrations ranging from 1-1000 nM (i.e., 1, 10, 100 or 1000 nM). When control wells neared confluence, cell number was determined for each well as an average of two readings on a ZM Coulter Counter. Results are expressed as the average cell number for each drug treatment group divided by the average cell number for the drug free control group (designated as % OF CONTROL) vs. the concentration of drug or analog.

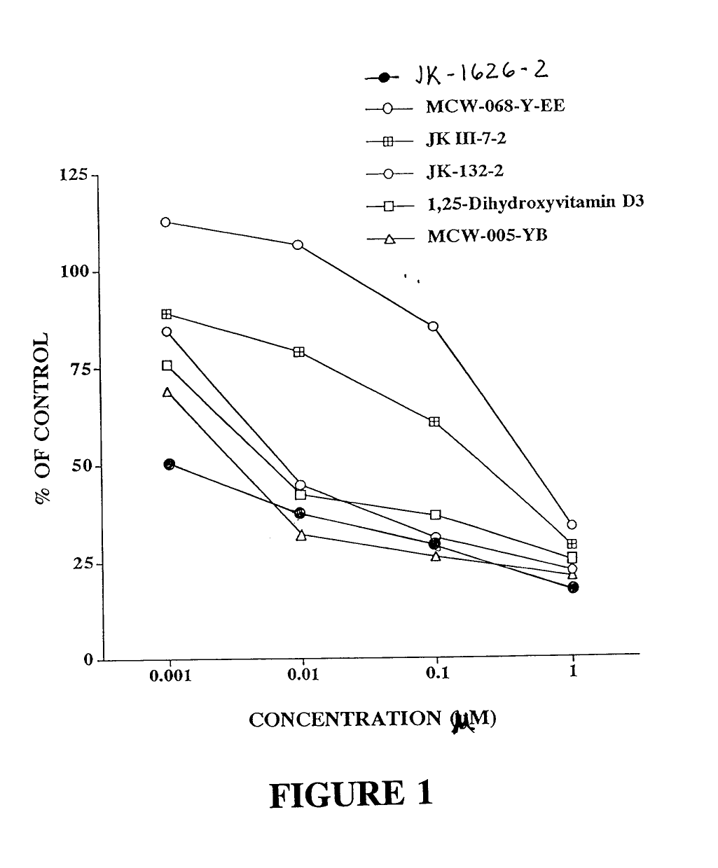

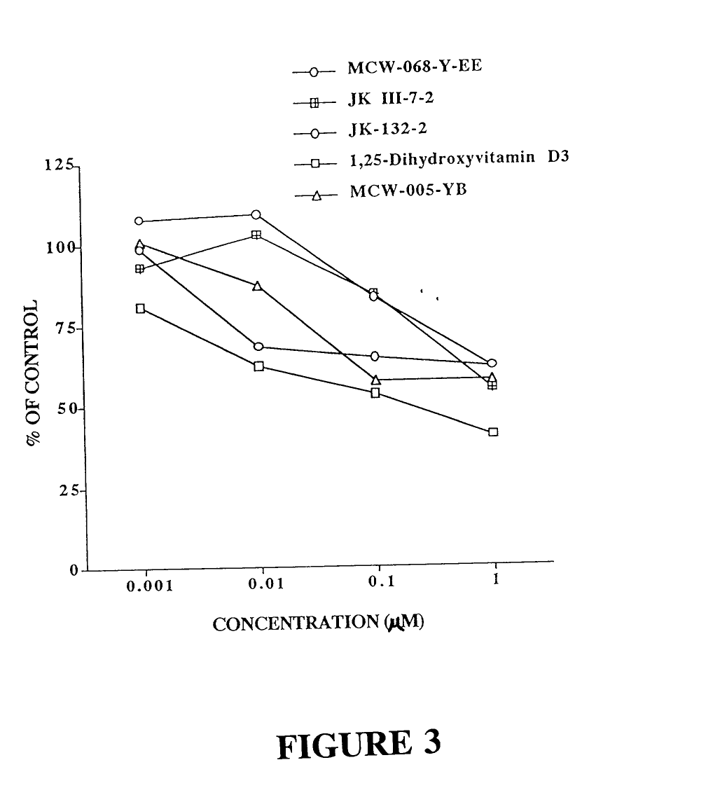

The results are shown in FIGS. 1-3 and summarized in Table 1. Five hybrid analogs, JK-III-7-2, MCW-068-Y-EE, JK-132-2, MCW-005-Y-B, and JK-1626-2, and 1,25 D₃ demonstrated significant antiproliferative activity at 10 nM against B16 and RENCA (p<0.03), at 100 nM against EMT6 (p<0.01), and at 1000 nM against CT26 (p<0.01, data not shown) (JK-1626-2 not yet tested against RENCA and CT26). MCW-005-YB and JK-1626-2 appeared to be the most potent analogs, consistently demonstrating antiproliferative activity similar to that of the parent compound.

TABLE 1
Antiproliferative effects of 1,25 D₃ and four hybrid analogs against Metastic Tumor Cell Lines

| | B16 | | RENCA | | EMT6 | |
|---|---|---|---|---|---|---|
| | | EC₅₀ Relative | | EC₅₀ Relative | | EC₅₀ Relative to |
| Drug | EC₅₀ (μM) | to 1.25 D₃ | EC₅₀ (μM) | to 1.25 D₃ | EC₅₀ (μM) | to 1.25 D₃ |
| 1.25 D₃ | 0.015 | 1 | 0.153 | 1 | 0.16 | 1 |
| MCW-005-YB | 0.004 | 0.29 | 0.070 | 0.46 | 1.26 | 7.88 |
| JK-132-2 | 0.019 | 1.28 | 0.271 | 1.78 | 3.36 | 21.02 |
| JK-III-7-2 | 0.164 | 10.92 | 0.359 | 2.35 | 10.17 | 63.62 |
| MCW-068-Y-EE | 0.671 | 44.66 | 0.343 | 2.24 | 8.80 | 55.06 |

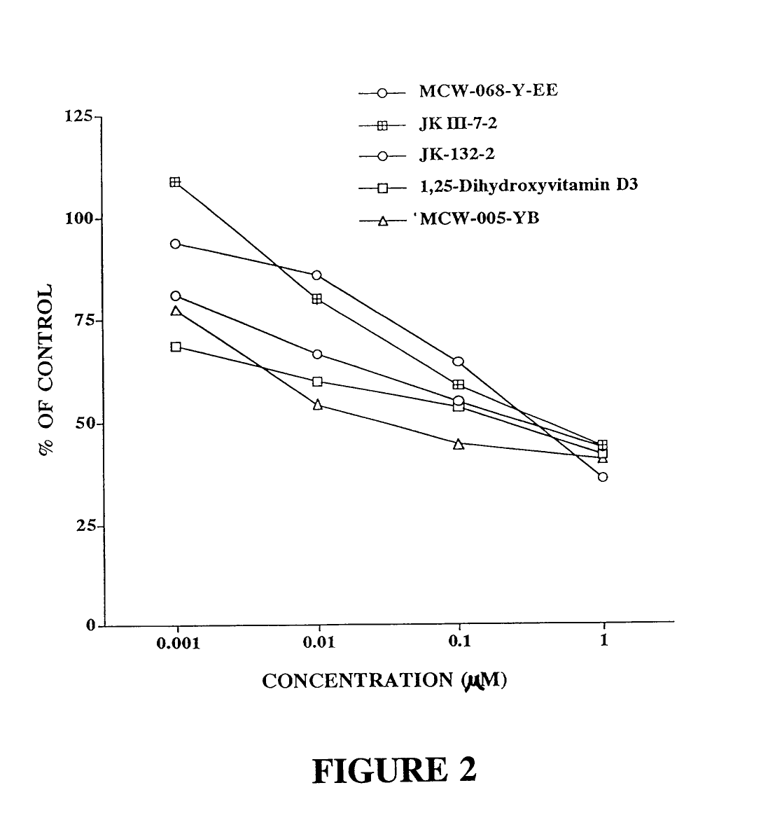

Table 1 shows the antiproliferative effects of 1,25 D₃ and four hybrid analogs against B16 (malignant melanoma), RENCA (renal cell carcinoma), and EMT6 (breast cell carcinoma). The concentration of each drug required to effect 50% inhibition of cell proliferation, designated as EC50, has been derived from the graphs shown in FIG. 2. The EC50 value relative to that of 1,25 D3 has also been calculated to allow for comparisons of drug potency.

Time Dependence Studies

In a series of exposure time dependence studies, B16 melanoma cells were trypsinized, suspended, and plated as before. After 24 hours of incubation original medium was removed and replaced with fresh medium containing either solvent or drug at a concentration of 10 nM in triplicate. Then at 1, 2, 10, 24, and 96 hours, the drug containing media was removed and replaced with fresh media containing only solvent. Then at 1, 2, 10, 24, and 96 hours the drug containing media was removed and replaced with fresh media containing only solvent. At the 96 hour time point, all groups were trypsinized and cell number was determined as before.

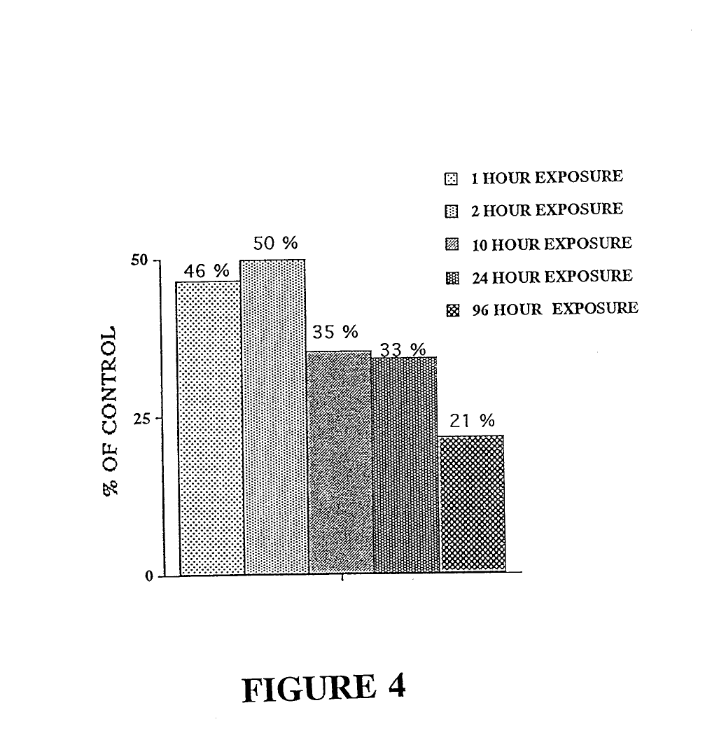

FIG. 4 demonstrates the exposure time dependent antiproliferative activity of 1,25 D3 at 10 μM against B16 malignant melanoma cells. Results are expressed as % of control, the mean cell number from 3 wells for each drug concentration divided by the mean cell number from 3 control wells receiving only solvent (0.4% isopropanol). These results demonstrate that the antiproliferative activity of 1,25 D₃ and its analogs is exposure time dependent, strengthening the rationale for sustained drug delivery as compared to bolus administration.

### Example 2

Testing the Trascriptional Upregulation of NGF by 1,25 D₃ and Hybrid Analog MCW-YB in Murine L929 Fibroblasts in vitro

In vitro studies were carried out to test the ability of 1,25 D₃ and the analog MCW-YB to upregulate the expression of NGF in murine L929 fibroblasts. L929 cells, obtained from ATCC (Rockville, Md.), were harvested from culture and plated at 50,000 cells per well on a Falcon 24 well tissue culture plate After 24 hours of incubation, culture media was removed from each well and replaced with serum free medium containing either 1,25 D₃ or MCW-YB at 100 nM or vehicle in triplicate. After 48 hours of incubation, the media from each well was quantitatively analyzed for NGF protein content using an enzyme linked immunosorbant assay (ELISA). The total NGF production per 50,000 cells was then determined using cell number values determined using a ZM Coulter Counter as before.

Treatment with the analog MCW-YB led to statistically significant (p<0.03) 40% increase in NGF expression compared to solvent controls. It is important to note that similar small but significant increases in NGF have been previously shown to be effective in the treatment of murine models of Alzheimer's disease.

### Example 3

Testing the Calcemic Activity of 1,25 D₃ and the Two Most Potent Hybrid Analogs, MCW-YB and JK-1626-2, in C57 Bl/6 Mice

Having established that the Posner analogs of 1,25 D₃ maintained their antiproliferative and transcriptional regulating activities in vitro, it was determined whether the most potent analogs MCW-YB and JK-1626-2 demonstrate substantially minimized calcemic activity in vivo. To test for calcemic activity, 1,25 D₃, MCW-YB, and JK-1626-2 were dissolved in a biocompatible solvent composed of 80% propylene glycol/20% phosphate buffered saline. Twenty-seven C57/B16 mice (n=3 per group), received daily intraperitoneal injections solution containing one of the three drugs at on of the following doses: 1, 10, or 100 mg/kg/day (corresponding to 0.02, 0.2. or 2 mg/day respectively). Nine animals received daily intraperitoneal injections of solvent only to serve as control. Animal weights were monitored daily at the time of injection. On day 7, all animals were sacrificed and blood was collected via cardiac puncture and quantitatively analyzed for ionized calcium content at the Critical Care Lab at Johns Hopkins Hospital.

Treatment with the parent compound at 1 and 10 mg/kg/day led to substantial toxic hypercalcemia, signified by substantial weight loss and dramatic rises in blood ionized calcium levels. The group receiving 1,25 D₃ at 100 mg/kg/day was so severely compromised that collection of sufficient blood samples for ionized calcium quantification was not possible. The hybrid analogs, however, were markedly less calcemic than the parent compound. Remarkably, absolutely no signs of toxic hypercalcemia were observed for the analog MCW-YB, i.e. no weight loss or significant rise in blood ionized calcium, at the 1, 10 and even the 100 mg/kg/day dosing regimens. No weight loss was observed following treatment with JK-1626-2 at 1 and 10 mg/kg/day as well. A small increase in blood ionized calcium was observed at the 10 mg/kg/day dosing regimen, but this was much less than the increase recorded for the parent compound at the same dose. Significant weight loss and a rise in blood ionized calcium were observed by day seven for the group receiving JK-1626-2 at 100 mg/kg/day, however both were significantly less severe than that observed for 1,25 D₃ at a 10× lower dose.

### Example 4

Incorporation of 1,25 D₃, MCW-YB, and JK-1626-2 into Biodegradable Polyanhydride Polymer Wafers and Demonstration of Controlled Drug Release in vitro

Polymer Formulation.

Hybrid analogs MCW-YB and JK-1626-2 were successfully loaded into biodegradable polyanhydride copolymer wafers composed of 1,3-bis(p-carboxyphenoxy) propane (CPP) and sebacic acid (SA) (20:80).

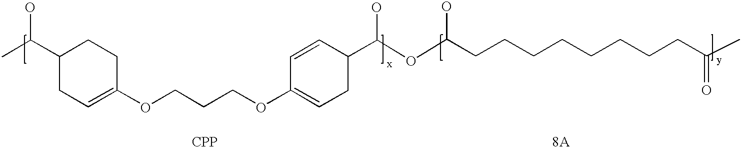

To prepare the drug/polymer formulations, polymer and drug (various % by weight loading) were co-dissolved in HPLC grade methylene chloride and the solution was dried overnight in vacuo. The resulting homogenous polymer formulation was compression molded into cylindrical wafers using a miniature custom made compression molding device similar to micro KBr dies available from Aldrich. This yielded 5 and 10 mg cylinders measuring 1.5 and 3 mm in diameter respectively and 0.5 mm in height. The polymer wafers were stored in anhydrous conditions for later use.

In Vitro Release Studies.

To determine the release kinetics of MCW-YB and JK-1626-2 from the pCPP:SA polymer formulations, 5 mg wafers were placed into 2 ml cryoware cryogenic mini-vials. To each vial was added 2 ml of a 30% ethanol/70% 0.01M phosphate buffered aqueous solution (pH 7.4). The ethanol was added to increase the solubility of the hydrophobic 1,25 D₃ analogs. Vials were incubated at 37° C. on an orbital shaker turning at 100 rpm. Periodically the buffer solution was removed and replaced with fresh buffer to approximate perfect sink conditions. The collected samples were analyzed for 1,25 D₃ analog content using quantitative high pressure liquid chromatography (HPLC) with a Beckmann system Gold (including an Autosampler 507, Programmable Solvent Module 126AA, and Programmable Detector Module 166 from Beckmann Instruments, San Roman, Calif.) controlled by Dell System 200 personal computer (Dell Computer Corporation, Austin, Tex.) and equipped with 4.6×250 mm Microsorb-MV C18 column (Rainin Instrument Company, Woburn, Mass.). The mobile phase consisted of acetonitrile/water (60:40), the flow rates were 1.8 (MCW-YB), and 2.25 (JK-1626-2) ml/min. UV detection was performed at wavelengths of 264 (MCW-YB) and 262 (JK1626-2) nM. Under these conditions the retention time was 9.6 min. for MCW-YB and 17.1 min. for JK-1626-2.

Continuous drug release (50.2% total) was demonstrated in vitro over a period of 110 hours for wafers loaded with MCW-YB at 2.1% (w/w). A series of polymers loaded with JK-1626-2 at loading doses ranging from 1 to 10% demonstrated continuous release for up to 200 hours. These results indicate that 1,25 D₃ analogs can be loaded into pCPP:SA (20:80) polymer formulations and released with maintained structural integrity in vitro. However, in the absence of ethanol, drug release will most likely occur more slowly, as would the case in vivo.

### Example 5

Determining the Highest Tolerated Dose of MCCW-YB and JK-1626-2 that can be Delivered to the Murine Flank and/or Brain via Biodegradable Polymer Wafers

Determination of the Highest Tolerated Doses in vivo

Using the hybrid analogs MCW-YB and JK-1626-2 loaded into pCPP:SA(20:80) wafers, the highest tolerated dose of 1,25 D₃ analogs that could be polymerically delivered to the murine brain without systemic toxicity due to hypercalcemia was determined. Polymer wafers with drug loadings ranging from 0.01% to 1% of each analog were prepared and implanted in the brains of C57 Bl/6 mice (n=4 per group). Animal weight loss (an established indicator of hypercalcemia) were monitored daily.

The highest tolerated doses for JK-1626-2 and MCW-YB were 0.1% and 1% respectively. The dramatic increase in tolerance for MCW-YB correlates well with the calcemic studies outlined in Example 3. Delivery of the parent compound, 1,25 D₃, to the brain of Sprague-Dawley rats using a mini-osmotic pump implanted intracerebroventricularly (i.c.v.) resulted in a rise in serum calcium after 6 days at the 60 ng/day dosing level. At 120 ng/day weight loss was observed, and reportedly at 240 ng/day the animals were “severely compromised” by day 6. In contrast, 10 mg polymer wafers loaded with 0.5% MCW-YB (50,000 ng of drug) implanted intracranially in 9 Sprague-Dawley rats caused no weight loss in the rats. Assuming a 20 day release period as is typical for the pCPP:SA (20:80) wafers, these animals were receiving about 2500 ng of the 1,25 D₃ analog MCW-YB per day (more than 10 times the dose of the parent compound reported to have caused severe hypercalcemic toxicity when delivered i.c.v.) and the study was carried out for twice as long (12 days). Aanalysis of blood samples collected via cardiac puncture at the time of serial sacrifice on days 1, 6, and even 12 showed no significant rise in blood calcium when compared to control animals receiving placebo wafers.

### Example 6

Testing the Hypothesis that Site-specific Polymeric Delivery of 1,25 D₃ Analogs can Result in Reduced Toxic Hypercalcemia

The hypercalcemic toxicity of polymerically delivered MCW-YB and JK-1626-2 was then compared to that of the parent compound, and used to test the hypothesis that site-specific polymeric delivery of 1,25 D₃ analogs can result in reduced toxic hypercalcemia. Twenty-four C57/Bl6 mice (n=3 per group) received intraflank or intracranial implantation of 5 mg pCPP:SA(20:80) polymer wafers loaded with no drug, 0.1% 1,25 D₃, 0.1% MCW-YB, or 0.1% JK-1626-2. Animal weights were monitored daily and blood was collected for quantitative ionized calcium analysis via cardiac puncture on day 7 post-implantation.

Both intraflank and intracranial implantation of polymer wafers loaded with 0.1% 1,25 D₃ led to severe toxic hypercalcemia as indicated by substantial weight loss and dramatic rises in blood ionized calcium levels compared to placebo controls. In stark contrast, animals treated with MCW-YB-loaded wafers showed no signs of toxic hypercalcemia following implantation at either locus. Intracranial polymeric delivery of the somewhat more calcemic analog, JK-1626-2, yielded no rise in blood ionized calcium levels; however, a significant increase was observed in animals receiving identical polymer wafers in the flank. This unique result with JK-1626-2 demonstrates that indeed site-specific polymeric delivery of 1,25 D₃ analogs to the murine brain minimizes hypercalcemic toxicity when compared to drug delivery to the flank. Similar results would be expected with 1,25 D₃ at a lower drug loading dose and with MCW-YB at a higher dose.

### Example 7

Testing the Efficacy of 1,25 D₃ Analog-loaded Polymer Wafers in the Treatment of Malignancy in vitro and in vivo

In vitro proliferation assays in which the 1,25 D₃ analogs were delivered from drug-loaded pCPP:SA (20:80) wafers were used to evaluate initially the therapeutic potential of 1,25 D₃ analog-loaded polymer wafers in the treatment of cancer. Cultured murine B16 malignant melanoma cells were trypsinized and plated at 5000 cells/well in Falcon 6 well tissue culture plates. After 24 hours to allow for cell attachment, 0.5 mg polymer wafers, created by sectioning a 5 mg wafer into 10 pieces, loaded with various amounts of MCW-YB or JK-1626-2, were added to cell culture media. Control wells received 0.5 mg placebo polymers. When control wells neared confluence, all wells were harvested and cell number was determined as before on a ZM Coulter Counter.

These drug-loaded polymers demonstrate potent antiproliferative activity in vitro against B16 malignant melanoma cells.

The therapeutic efficacy of this strategy was also tested in vivo. A solid tumor flank model was developed in which 50,000 EMT6 breast carcinoma cells harvested from culture are injected subcutaneously in Balb-C mice; after nine days, palpable solid flank tumors are observed (MCW-005-YB EMT6 Breast Carcinoma Model). In the first study using this model, tumors were measured on day 9 and animals were randomized into two treatment groups. Seven mice received placebo polymer wafers and 7 mice received wafers loaded with MCW-YB at half the highest tolerated intracranial dose (0.5% w/w) in the flank. Tumor volume was measured every other day in a blinded fashion using venier calipers and animal weights were periodically determined.

The results indicate that MCW-YB, when delivered locally from pCPP:SA wafers, inhibits the growth of EMT6 solid tumors. However, due to low numbers of animals included in each group and unexpected lethal toxicity observed in the treatment arm the results were not statistically significant.

In conclusion, these studies demonstrate the therapeutic potential of controlled release polymers loaded with anticalcemic analogs of 1,25 D₃, in the treatment of a variety of malignancies as well neurodegenerative disorders such as Alzheimer's disease.

We claim:

1. A controlled or sustained release formulation comprising
vitamin D3 or an analog thereof having antiproliferative activity, and
a polymeric matrix.

2. The formulation of claim 1 wherein the vitamin D3 or analog is present in a dosage effective to inhibit proliferation or to cause toxicity of malignant cells.

3. The formulation of claim 1 wherein the vitamin D3 or analog is present in a dosage effective to induce expression of nerve growth factor.

4. The formulation of claim 1 wherein the formulation comprises a vitamin D3 analog in a polymeric matrix.

5. The formulation of claim 4 wherein the vitamin D3 analog has the formula 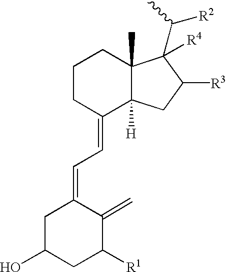
wherein R¹ is —OH or CH₂OH, R2 is a C4-6 chain or a C4-6 alkoxy chain, wherein the chain includes one or more substituents selected from the group consisting of hydroxyl groups, preferably tertiary hydroxyl groups, alkene groups, alkyne groups, alkyl groups, preferably methyl and ethyl, and ketones, and R3 and R4 are either H or together form a double bond.

6. The formulation of claim 4 wherein the analog has less calcemic activity than vitamin D3.

7. The formulation of claim 5 wherein the analog is selected from the group consisting of

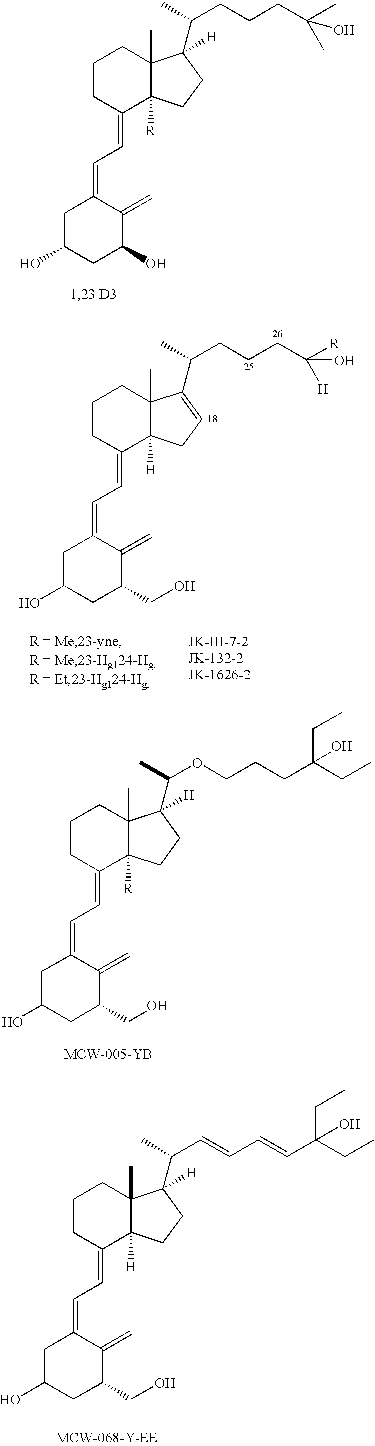

8. A method of treating a patient to inhibit tumor viability or proliferation comprising administering to the patient at a site where tumor is found any of the formulations of claims 1-7.

9. A method of treating a patient to induce expression of nerve growth factor comprising administering to the patient at a site in the central nervous system any of the formulations of claims 1-7.

10. The method of claim 9 wherein the patient suffers from a neurodegenerative disorder and the formulation is administered to the brain.
